(19) United States
(12) Patent Application Publication
(10) Pub. No.: US 2022/0355074 A1
(43) Pub. Date: Nov. 10, 2022

(54) NASOGASTRIC TUBE POSITIONING SYSTEM AND DETECTION METHOD

(71) Applicants: NASOTRAK MEDICAL PTE. LTD., Singapore (SG); TAN TOCK SENG HOSPITAL PTE LTD, Singapore (SG); ALEXANDRA HEALTH PTE. LTD., Singapore (SG)

(72) Inventors: Khai Pang LEONG, Singapore (SG); Ravinder Singh SACHDEV, Singapore (SG); Wern Hsien BIN, Singapore (SG); Chee Mun Eric LOH, Singapore (SG); Teck Hui YONG, Singapore (SG)

(73) Assignees: NASOTRAK MEDICAL PTE. LTD., Singapore (SG); TAN TOCK SENG HOSPITAL PTE LTD, Singapore (SG); ALEXANDRA HEALTH PTE. LTD., Singapore (SG)

(21) Appl. No.: 17/627,639

(22) PCT Filed: Jul. 15, 2020
(86) PCT No.: PCT/SG2020/050414

§ 371 Date: Jan. 14, 2022

(30) Foreign Application Priority Data
Jul. 15, 2019 (SG) 10201906548V

(51) Int. Cl. A61M 25/01; A61J 15/00

(52) U.S. Cl. CPC A61M 25/0108; A61J 15/0003; A61J 15/0088; A61J 15/0073; A61M 2205/502

(57) ABSTRACT

The invention relates to a nasogastric tube (100) comprising a nasogastric tubing (102) having first and second ends (102a, 102b), a power supply part (106) located at or adjacent the first end, and a transducer part (104) located at or adjacent the second end for transmitting or receiving an ultrasound based signal. Also disclosed is a positioning system comprising the said nasogastric tube, and a control module comprising an at least one detection portion and a processor.

### FIELD

The present disclosure generally relates to nasogastric tubes, and in particular to a nasogastric tube positioning system and a method of constructing the nasogastric tube and a detection method in association with the nasogastric tube.

### BACKGROUND

The following discussion of the background to the invention is intended to facilitate an understanding of the present invention only. It should be appreciated that the discussion is not an acknowledgement or admission that any of the material referred to was published, known or part of the common general knowledge of the person skilled in the art in any jurisdiction as at the priority date of the invention.

Currently, after a nasogastric (NG) tube has been inserted in a body, the use of X-ray based or the use of pH aspirate-based techniques, for the purpose of verifying NG tube placement, would be common. There are however disadvantages associated with the use of these existing techniques. As these techniques require suitably qualified and trained personnel to conduct them, they cannot be used outside of a hospital or medical clinic environment. In recent years, new methods of NGT insertion and placement confirmation have surfaced. However, there are still incidents of wrong placement leading to detrimental effects.

The present disclosure contemplates that it would be desirous to consider one or more other techniques for verifying NG tube placement so that more options for such a purpose can be available.

An object of the invention is to ameliorate one or more of the above-mentioned difficulties.

### SUMMARY

According to an aspect of the present disclosure, there is provided a nasogastric tube comprising a nasogastric tubing having first and second ends, a power supply part located at or adjacent the first end, and a transducer part located at or adjacent the second end for transmitting or receiving an ultrasound-based signal.

In some embodiments, the transducer part may be sealed within a distal tip located at the second end.

In some embodiments, the distal tip may be at least substantially dome shaped.

In some embodiments, the power supply part may be a power connector for connecting a power supply to the transducer part.

In some embodiments, the power supply part may be a power supply for supplying power to the transducer part.

In some embodiments, the tubing may comprise a first lumen extending along a length of the tubing, and one or more apertures extending through the tubing into the first lumen at or adjacent the second end thereof, the first lumen allowing fluids including food and medicine to be transferred therethrough and from the one or more apertures.

In some embodiments, the tubing may comprise a second lumen extending along the length of the tubing and separate from the first lumen, the second lumen supporting a wiring system therein electrically connecting the power supply part to the transducer part.

In some embodiments, the wiring system may be a twisted wire pair.

In some embodiments, a radiopaque line may extend along the length of the tubing.

According to another aspect of the present disclosure, there is provided a nasogastric tube positioning system comprising a nasogastric tube as described above, and a control module comprising an at least one detector portion and a processor, wherein ultrasound-based signals are transmittable between the transducer part and the at least one detector portion, the processor being adapted to record the time taken for the ultrasound-based signal to travel between the transducer part and the at least one detector portion, and to calculate a distance between the transducer part and the at least one detector portion to thereby locate the transducer part.

In some embodiments, the control module may comprise two said detector portions located a fixed distance apart.

In some embodiments, the control module may be a handheld scanner having a housing for accommodating the detector portions.

In some embodiments, the nasogastric system may further comprise a power supply within the housing for providing electrical power to the transducer part and the detector portions.

In some embodiments, the nasogastric system may further comprise a display screen for showing when the detector portions are shaped equidistant from the transducer part.

In some embodiments, the nasogastric system may further comprise a device for displaying a light or audio signal when the detector portions are shaped equidistant from the transducer part.

According to a further aspect of the present disclosure, there is provided a detection method for a nasogastric tube positioning system as described above, comprising transmitting an ultrasound-based signal between the transducer part located at an end of the nasogastric tube and at least two said detector portions, recording the time taken for the ultrasound-based signal to travel between the transducer part and each of the detector portions, calculating the distance between the transducer part and each of the detector portions, and determining when the detector portions are equidistant from the transducer part to thereby locate the transducer part.

In some embodiments, the detection method may comprise selecting the transducer part as a transmitter of the ultrasound-based signal, with the detector portions being ultrasound receivers.

In some embodiments, the detection method may comprise selecting the detector portions as transmitters of the ultrasound-based signal, with the transducer part being a ultrasound receiver.

In some embodiments, the detection method may comprise scanning an abdomen of a patient in a first direction initially until the detector portions are equidistant from the transducer part, and subsequently repeating the scan in a direction that is 90 degrees from the first direction.

According to yet another aspect of the present disclosure, there is provided a method of manufacturing a nasogastric tube, comprising providing a tubing having the first and second ends, coupling a power supply part to the first end thereof, and a transducer part to the second end thereof, the transducer part being embedded into a distal tip of the second end.

Other aspects and features will become apparent to those of ordinary skill in the art upon review of the following description of specific embodiments in conjunction with the accompanying figures.

### BRIEF DESCRIPTION OF THE DRAWINGS

In the figures, which illustrate, by way of example only, embodiments of the present disclosure,

FIG. 1 shows a nasogastric tube positioning system having a nasogastric tube including a transducer part configurable to generate and transmit one or more ultrasound-based signals and a control module, according to an embodiment of the disclosure;

FIG. 2a and b respectively show a partial side and cross-sectional view of a nasogastric tube according to another embodiment of the present disclosure;

FIG. 3a and FIG. 3b show an exemplary manner in which the ultrasound-based signal(s) can be processed, according to an embodiment of the disclosure;

FIG. 4 is a flow chart showing the processing steps used in the control module according to the present disclosure;

FIG. 5 is a flow chart showing the operational steps of the nasogastric tube positioning system according to the present disclosure;

FIG. 6a shows a construction method in association with the nasogastric tube of FIG. 1, according to an embodiment of the disclosure; and

FIG. 6b shows a detection method in association with the nasogastric tube of FIG. 1, according to an embodiment of the disclosure.

### DETAILED DESCRIPTION

Throughout this document, unless otherwise indicated to the contrary, the terms “comprising”, “consisting of”, “having” and the like, are to be construed as non-exhaustive, or in other words, as meaning “including, but not limited to”.

Furthermore, throughout the specification, unless the context requires otherwise, the word “include” or variations such as “includes” or “including” will be understood to imply the inclusion of a stated integer or group of integers but not the exclusion of any other integer or group of integers.

The present disclosure contemplates that it can be useful to utilize ultrasound based techniques/technologies for the purpose of verifying placement of a nasogastric (NG) tube after the NG tube has been inserted/positioned in a body (e.g., human body).

Specifically, the present disclosure contemplates that it can be useful to utilize ultrasound based techniques/technologies for the purpose of verifying placement of at least a portion of a nasogastric (NG) tube that has been inserted/positioned in a body (e.g., human body).

The present disclosure further contemplates that the use of ultrasound for the purpose of verifying NG tube placement has not previously been considered due to one or more technical barriers and a solution is yet available to overcome such technical barrier(s).

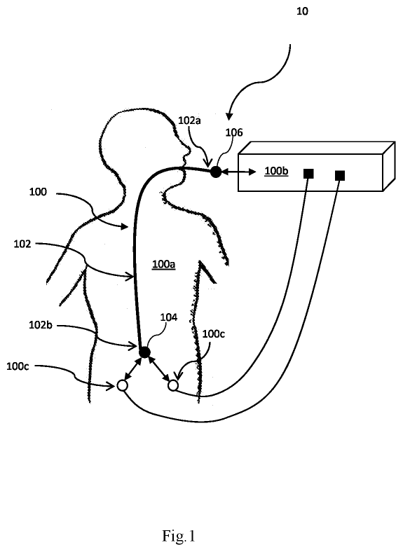

As will be discussed hereinafter with reference to FIG. 1 to FIG. 6b, the present disclosure contemplates at least one possible manner in/by which the use of ultrasound-based techniques/technologies can be facilitated in the context of a nasogastric tube.

Referring to FIG. 1, there is shown a nasogastric tube positioning system 10 comprising a nasogastric (NG) tube 100 and a control module 100b, according to an embodiment of the disclosure. As shown, the NG tube 100 can be placed/located within a body 100a. Moreover, the NG tube 100 can be coupled to the control module 100b which can include one or more detector portions 100c. The control module 100b will be discussed later in further detail. Additionally, it is to be appreciated that the NG tube 100 and the control module 100b can, in effect, form/constitute a system (i.e., the system can include the NG tube 100 and the control module 100b).

Specifically, the NG tube 100 can be associated with ultrasound-based techniques/technologies in that the NG tube 100 position, when within a body 100a, can be verified/determined by manner of ultrasound. Specifically, position of at least a portion of the NG tube 100 that is within the body 100a can be verified/determined by manner of ultrasound-based techniques/technologies.

The nasogastric tube 100 can include a tubing 102, a transducer part 104 and a power supply part 106. Moreover, the tubing 102 can be shaped and dimensioned in a manner so as to be capable of carrying a transducer part 104 and a power supply part 106. Furthermore, the transducer part 104 can be coupled to the power supply part 106. Coupling between the transducer part 104 and the power supply part 106 can be based on one or both of wired coupling and wireless coupling.

The tubing 102 can include a first end 102a and a second end 102b. The first end 102a and the second end 102b can define the extremities of the tubing 102. Specifically, the first and second ends 102a/102b can be opposing ends of the tubing 102.

In one example, the tubing 102 can be in the form of an elongated structure. In a more specific example, the tubing 102 can be in the form of a flexible elongated structure. In yet a more specific example, the tubing 102 can be a flexible elongated structure made of material such as clear thermoplastic polyurethane (TPU) or polyvinyl chloride (PVC).

Additionally, as shown, when inserted in the body 100a, one end (e.g., the first end 102a) of the NG tube 100 can be nearer to the external of the body 100a as compared to another end (e.g., the second end 102b) of the NG tube 100. In a specific example, when the NG tube 100 has been inserted in the body 100a, the first end 102a can be visually perceivable outside of the body 100a whereas the second end 102b is within the body 100a (e.g., within the abdominal portion of the body 100a).

As earlier mentioned, the tubing 102 can be shaped and dimensioned in a manner so as to be capable of carrying a transducer part 104 and a power supply part 106. Further earlier mentioned, the transducer part 104 can be coupled to the power supply part 106.

Specifically, the transducer part 104 can be carried by the tubing 102 nearer to its second end 102b (i.e., relative/compared to the first end 102a) whereas the power supply part 106 can be carried by the tubing 102 nearer to its first end 102a (i.e., relative/compared to the second end 102b). In one example, the transducer part 104 can be carried by the tubing 102 at the second end 102b whereas the power supply part 106 can be carried by the tubing 102 at its first end 102a. In one embodiment, the transducer part 104 can be considered to be embedded at a distal end (e.g., the second end 102b) of the NG tube 100.

The transducer part 104 can, for example, be an ultrasound-based transmitter. Specifically, the transducer part 104 can be configured to transmit one or more ultrasound-based signals. More specifically, the transducer part 104, when activated, can be configured to transmit one or more ultrasound-based signals. In one embodiment, the transducer part 104 can be activated by manner of receiving power which can be communicated from the power supply part 106. In this regard, the transducer part 104 can, for example, be considered to be a power activated transmitter. In a more specific example, the transducer part 104 can be considered to be a power activated ultrasound-based transmitter. As mentioned, power to the transducer part 104 can be communicated from the power supply part 106.

The power supply part 106 can be based on one or both of a standalone based power supply scheme and a dependent based power supply scheme.

In regard to the standalone based power supply scheme, the power supply part 106 can be shaped and dimensioned in a manner so as to be capable of carrying a standalone type power source. A standalone type power source can, for example, be a battery. In a more specific example, power from a battery (i.e., carried by the power supply part 106) can be communicated to the transducer part 104 for activating the transducer part 104. In this regard, the power supply part 106 can correspond to a structure such as a battery holder, according to an embodiment of the disclosure.

In regard to the dependent based power supply scheme, the power supply part 106 can be configured to receive power from an external power source (not shown) and regulate the received power. Regulated power can subsequently be communicated to the transducer part 104 for activating the transducer part 104. In this regard, the power supply part 106 can correspond to a regulator (e.g., a voltage regulator) which can be configured to receive power from an external power source, according to an embodiment of the disclosure. The regulator can be coupled to an external power source by manner of one or both of wired coupling and wireless coupling.

Moreover, in regard to the dependent based power supply scheme, the power supply part 106 can be configured to receive power from an external power source (not shown). Power received from the external power source can be communicated to the transducer part 104 for activating the transducer part 104. In this regard, the power supply part 106 can correspond to a coupling portion which can be coupled to an external power source. The coupling portion can be coupled to an external power source by manner of one or both of wired coupling and wireless coupling.

In this regard, it is to be appreciated that the power supply part 106 can be coupled to an external power source by manner of one or both of wired coupling and wireless coupling, according to one embodiment of the disclosure.

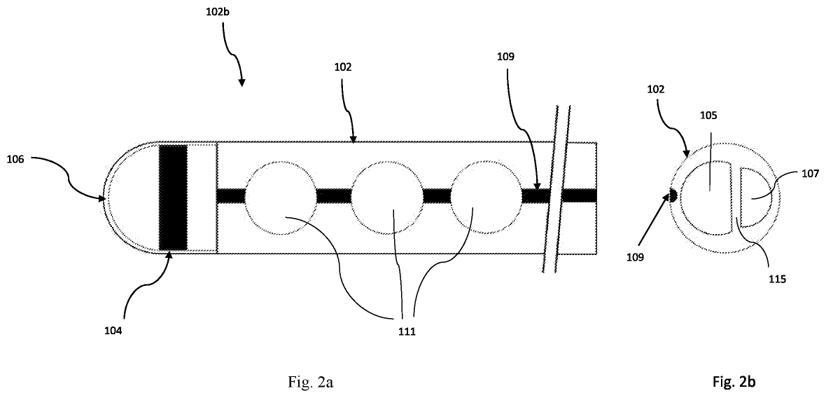

FIG. 2a and b shows an alternative embodiment of the NG tube 100 according to the present disclosure. The same reference numerals are used for corresponding features of this embodiment for clarity reasons. FIG. 2a shows in detail the second end 102b of the NG tube 100, with the transducer part 104 being located at the peripheral end of the tubing 102. The transducer part 104 can be embedded and sealed within a dome shaped distal tip 106. The tubing 102 further includes lumens 102,107 as shown in FIG. 2b, with each lumen extending along the entire length of the tubing 102. The second end 102b includes a number of apertures 111 which are in fluid communication with the first lumen 105. Food and medicine can be transferred though the first lumen105 and apertures 111 for administering to the patient. The second lumen 107 is an electrical conduit through which can be run electric wires (not shown) to allow power to be supplied to the transducer part 104. A twisted wire pair may for example be used for this purpose as this minimizes any electric and/or magnetic effects on the electrical wiring. The first and second lumens 105,107 are separated by a dividing wall 115 to ensure that the fluid passing through the first lumen 105 do not interreact with the electrical wiring within the second lumen 107. Nevertheless, the electrical wiring may be insulated to prevent electrical risk or short circuiting if there is any breakage within the dividing wall leading to the leaking of fluid into the second lumen 107.

The tubing 102 may for example be designed to have a 14 Fr size (with a 4.7mm outer diameter), and may be made from a flexible material resistant to gastric acid for an extended period of time. It is however to be appreciated that the present disclosure is not limited to this tubing size, and that the use of alternative tubing sizes, for example from 12 to 18 Fr, is also envisaged. A flexible thermoplastic polyurethane (TPU) material may for example be used as this material can handle exposure to gastric acid for a period of 2 weeks to 1 month. A radiopaque line 109 may also optionally extend along the length of the tubing 102 as this facilitates the use of X-ray detection of the NG tube 100 if alternatively used.

The transducer part 104 may be made from a piezoelectric or other ultrasound generating material such as magneto strictive materials. In the present disclosure, the transducer part 104 may be the form of a disc and may have a diameter of up to around 3.00 mm. It is also envisaged that the transducer part 104 have other forms. For example, the transducer part 104 may be cylindrical or spherical in shape. The transducer part 104 can be entirely embedded and sealed by a biocompatible glue at the distal tip 106. Alternatively, the transducer part 104 can be integrally moulded into the TPU material forming the tubing 102. It is however also envisaged that the transducer part 104 be secured to an outer surface of the tubing 102 and covered with a thin film of acid resistant material such as the above mentioned TPU material. This ensures that the transducer part 104 is sealed and shielded from interaction with the surrounding fluids. Also, this helps to ensure that there are no airgaps surrounding the transducer part 104 that can affect the ultrasound-based signal transmission therefrom.

As earlier mentioned, the NG tube 100 can be coupled to a control module 100b. Further, as earlier mentioned, the control module 100b can include one or more detector portions 100c.

Specifically, the NG tube 100 can be configured to communicate with the control module 100b.

More specifically, the NG tube 100 can be coupled to the control module 100b so that the ultrasound-based signal(s) communicated from the transducer part 104 can be received by the control module 100b. The NG tube 100 and the control module 100b can be coupled by manner of one or both of wired coupling and wireless coupling.

Yet more specifically, ultrasound-based signal(s) communicated from the transducer part 104 can be received by the detector portion(s) 100c. The received ultrasound-based signal(s) can be further communicated for processing in a manner so as to verify the NG tube 100 position (i.e., within the body 100a).

In one embodiment, the control module 100b can be configured to process the received ultrasound-based signal(s) in a manner so as to verify the NG tube 100 position (i.e., within the body 100a). In this regard, the control module 100b can include a processor (not shown) which can be coupled to the detector portion(s) 100c and which can be configured to process the received ultrasound based signal(s) in a manner so as to verify the NG tube 100 position (i.e., within the body 100a).

In another embodiment, the control module 100b can be configured to further communicate the received ultrasound based signal(s) to one or more computers (not shown) for processing in a manner so as to verify the NG tube 100 position (i.e., within the body 100a). The control module 100b can, for example, be coupled to the computer(s) by manner of one or both of wired coupling and wireless coupling.

In yet another embodiment, the control module 100b can be configured to process the received ultrasound-based signal(s) and further communicate the received ultrasound-based signal(s) to one or more computers (not shown) for processing.

Generally, the received ultrasound-based signal(s) can be processed in a manner so as to verify the NG tube 100 position (i.e., within the body 100a). In a specific example, the received ultrasound based signal(s) can be processed in a manner so as to verify/determine the position of at least a portion of the NG tube 100 (e.g., the second end 102b) that is within the body 100a.

In a general example, the control module 100b can be configured to generate one or both of at least one audio based output signal and at least one graphical based output signal for indicating the NG tube 100 position (i.e., within the body 100a). The audio-based output signal(s) can be capable of being audibly perceived and the graphical based output signal(s) can be capable of being visually perceived. In this regard, it is to be appreciated that the control module 100b can further include at least one output portion (e.g., a speaker driver and/or a screen).

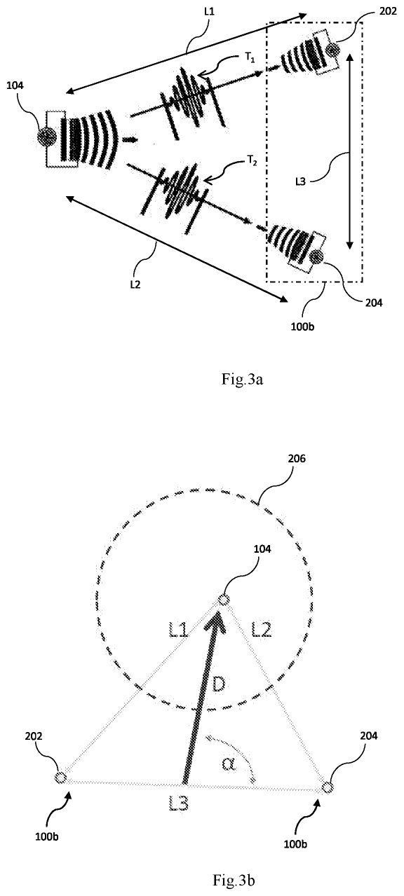

An exemplary manner in which the received ultrasound based signal(s) can be processed (i.e., in a manner so as to verify the NG tube 100 position when within the body 100a) will be discussed with reference to FIG. 3a and FIG. 3b hereinafter.

As shown in FIG. 3a and FIG. 3b, the control module 100b can include a first detector portion 202 and a second detector portion 204. The first and second detector portions 202,204 can be positioned relative to the transducer part 104 which is located within the body 100a. Specifically, the first and second detector portions 202,204 can be configured to receive the ultrasound-based signal(s) communicated from the transducer part 104. Moreover, the transducer part 104 can be associated with a detection range/area 206 (e.g., a certified detection range).

The present disclosure contemplates that time taken for the ultrasound-based signal(s) to travel from the transducer part 104 to each of the first and second detector portions 202,204 can be determined. Moreover, the ultrasound-based signal(s) can be associated with speed (e.g., speed of ultrasound in water).

Based on traveling time of the ultrasound-based signal(s), distance (i.e., “L”) between the transducer part 104 and a detector portion 202,204 can be determined. Specifically,
“L” (i.e., distance in millimetres)=“T” (i.e., traveling time in milliseconds) multiplied by “C” (i.e., speed of ultrasound in water)

Based on time taken for the ultrasound-based signal(s) to travel from the transducer part 104 to the first detector portion 202, distance (i.e., labeled as “L1”) between the transducer part 104 and the first detector portion 202 can be determined. Specifically,
“L1” (i.e., distance, as between the transducer part 104 and the first detector portion 202, in millimetres)=“T₁” (i.e., traveling time, as between the transducer part 104 and the first detector portion 202, in milliseconds) multiplied by “C” (i.e., speed of ultrasound in water)

Similarly, based on time taken for the ultrasound based signal(s) to travel from the transducer part 104 to the second detector portion 204, distance (i.e., labeled as “L2”) between the transducer part 104 and the second detector portion 204 can be determined. Specifically,
“L2” (i.e., distance, as between the transducer part 104 and the second detector portion 204, in millimetres)=“T₂” (i.e., traveling time, as between the transducer part 104 and the second detector portion 204, in milliseconds) multiplied by “C” (i.e., speed of ultrasound in water)

Moreover, distance (i.e., labeled as “L3”) between the first and second detector portions 202,204 can be determined (e.g., measured) after they have been positioned relative to the transducer part 104.

Thereafter, the distance “D” and angle “a” can be determined (e.g., by manner of calculation).

The present disclosure contemplates that, in one embodiment, by manner of using a plurality of detector portions (i.e., two or more detector portions 202/204), position of the NG tube 100 (e.g., the second end 102b) within the body 100a can be pinpointed accurately by way of angulation.

In another embodiment, the present disclosure contemplates that the use of only one detector portion may possibly suffice for the purpose of pinpointing position of the NG tube 100 (e.g., the second end 102b) within the body 100a.

Generally, the underlaying principal behind this solution is based on known speed that ultrasound can travel across different materials (e.g., water), calculating/measuring the time taken for the ultrasound based signal(s) to travel from the transducer part 104 to the detector portion 202,204 and, thereafter, estimating the distance between the transducer part 104 and the detector portions 202,204.

Additionally, in an exemplary general context, the NG tube 100 can be capable of transmitting ultrasound-based signal(s) (i.e., via the transducer part 104) after being powered (i.e., via the power supply part 106) with an optimum or controlled amount of power. The detector portion(s) 202,204 (i.e., positioned outside of the body 100a) can be configured to detect the ultrasound-based signal(s). Upon successful detection, the aforementioned control module 100b (i.e., which can correspond to a detection device) can be configured to provide at least one indication (audio based indication and/or visual based indication) of the location (i.e., within the body 100a) of the NG tube 100.

Moreover, in the above mentioned exemplary general context, the transducer part 104 can, in one embodiment, be considered to be embedded within the NG tube 100. Specifically, the transducer part 104 can, for example, be encased in a biocompatible material and powered (e.g., externally) via the power supply part 106. The transducer part 104 can, for example, be coupled to the power supply part 106 by manner of a built-in wire (i.e., The NG tube 100 can carry a built-in wire which can electrically couple the transducer part 104 and the power supply part 106).

The present disclosure contemplates that the physics behind the transmission of the ultrasound based signal(s) through the body 100a and the detection thereof (i.e., externally) can, for example, be analysed through bench simulation (e.g., a simulation type software carried by the control module 100b) to optimise accuracy and reliability of detection of the NG tube 100 location (i.e., within the body 100a).

The present disclosure further contemplates that a challenge faced is to ensure that the aforementioned system can be suitable for use by a broad spectrum of users (e.g., patients) with different physical attributes.

Specifically, the present disclosure contemplates that the aforementioned system is to be configured to function in a reliable manner as long as the transducer part 104 is within the aforementioned detection range/area 206 (e.g., within the abdominal area of a person). Therefore, regardless of variance in distance (i.e., of the transducer part 104 within the stomach of a person to the detector portion(s) 202,204 owing to variance in physical attribute(s) of a broad spectrum of users, reliable detection can be possible as the aforementioned system is configured to detect (i.e., the aforementioned ultrasound-based signal(s)) based on the detection range/area 206 being, for example, a base reference (i.e., detection can be confined to a pre-defined target area as defined by the detection range/area 206 so as to reduce the possibility of errors encountered in view of variance in physical attribute(s)). The present disclosure contemplates that an overlaying template (not shown) can be designed for use together with the aforementioned system to guide a user in positioning the detector portion(s) 202,204 on the abdomen (i.e., within the detection range/area 206).

The present disclosure contemplates the possibility of a detection range of up to 20 cm, or up to 30 centimeters (cm) for obese users. The present disclosure further contemplates the possibility of broad-based adoption (i.e., from medical facilities such as hospitals to homes) as the aforementioned system can be considered to be cost effective and/or user friendly.

In the above described arrangement, the transducer part 104 is adapted to transmit an ultrasound-based signal for detection by the detector portions 202,204. It is however also possible for the detector portions 202, 204 to be configured to be transmitters of the ultrasound-based signals, while the transducer part 104 is configured to be a detector of the ultrasound-based signals transmitted by the detector portions 202,204. The control module 100b can still operate using the same principles as previously described to determine the distances between the transducer part 104 and detector portions 202,204. It is also envisaged that more than two detector portions be used by the control module 100b to provide a three-dimensional map for locating the transducer part 104. Alternatively, a single detector portion could be used by the control module 100b if it is only necessary to obtain a general location of the transducer part 104.

The frequency of the pulses of the ultrasound system used in the nasogastric tube positioning system 10 according to the present disclosure may be greater than 20 kHz. The frequency may also more specifically be in the range of 60 kHz to 4 MHz which can facilitate the operation of the present system. It is however to be appreciated that the present disclosure is not limited to operation in these frequency ranges, an could operate at other frequencies.

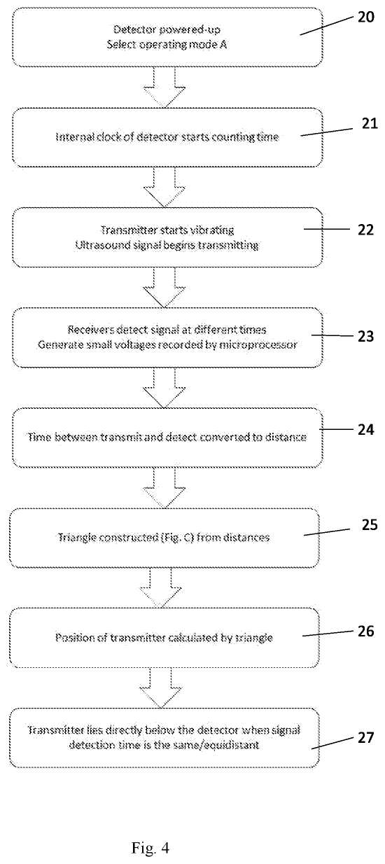

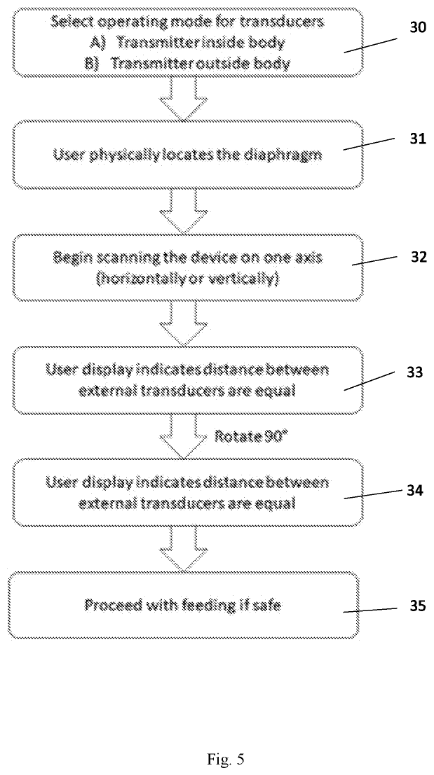

FIG. 4 is a flowchart showing the various processing steps of the control module 100b according to the present disclosure when the transducer part 104 has been selected as the ultrasound signal transmitter, and the detector portions 202,204 have been selected as the receivers of that ultrasound-based signal. The process steps are as follows:
a) The control module 100b is powered on and provides electrical power to the detector portions 202,204. Power is also provided to the transducer part 104. Operating mode A (see FIG. 5) can be selected where the transducer part 104 is the ultrasound transmitter (Step 20).
b) The control modules 100b has an internal clock that starts to count the time (step 21).
c) The transducer part 104 starts vibrating to thereby transmit an ultrasound-based signal though the body 100a towards the detector portions 202,204 (Step 22).
d) The detector portions 202,204 can then pick up the ultrasound-based signal at different times at the microsecond level if they are not equidistant from the transducer part 104. The detector portions 202,204 then generate a small voltage which is amplified and recorded by the processor(not shown) of the control module 100b as time value data (Step 23).
e) The processor then processes this time value data to convert them into distance values using the average speed of sound in body tissue (Step 24).
f) The processor will then use these distance values to form a triangle as shown in FIG. 3a (Step25).
g) The position of the transducer part 104 can then be mapped in two dimensions using this triangle (Step 26).
f) The position of the transducer part 104 will be found when the created triangle is equidistant distant because the same signal detection time is obtained from each detector portion 2002,204. The transducer part 104 will then be positioned directly below or in front of the control module 100b (Step 27).

According to another embodiment of the nasogastric tube positioning system 10 according to the present disclosure, the control module 100b may be in the form of a handheld and portable scanner (not shown). The detector portions 202,204 may be supported within the housing of the scanner. Alternatively, the detector portions 202,204 may be freely supported at the end of electric wires extending from the scanner housing. The power source of the scanner, which can for example be batteries, can also act as the power source for the transducer part 104, and an electrical connector may be provided on the scanner for connecting to the power supply part 106. The transducer part 104 can therefore be disconnected from the power supply when not being used for safety reasons. It is however also envisaged that the transducer part 104 be wirelessly supplied with power eliminating the need for a wiring system to be provided within the NG tube 100.

FIG. 5 is a flow chart showing the operational steps of the control module 100b when in the form of such a handheld scanner. The operational steps for a caretaker of a patient using the scanner are as follows:
a) There are two operational modes that can be initially set for the scanner by the caretaker. The transducer part 104 can be selected as the ultrasound transmitter within the body, with the detector portions 202,204 acting as ultrasound receivers, in operational mode A. Alternatively, the detector portions 202,204 can be selected as the ultrasound transmitters, with the transducer part 104 acting as the ultrasound receiver, in operational mode B (Step 30).
b) The location of the diaphragm of the patient is then determined using conventional medical techniques involving feeling below the ribcage of the patient (Step 31).
c) The caretaker can then commence scanning of the abdomen of the patient below their diaphragm in either a vertical or horizontal direction initially (these will be the directions when the patient is in a standing position, and will therefore correspond to directions along the length of and lateral across the patient respectively when the patient is lying down) (Step 32)
d) The scanner may for example include a display screen for displaying how centred the scanner is over the transducer part 104, with the scanner being centred when the same distance value is provided between the transducer part 104 and each detector portion 202,204. The scanner may alternatively or additionally have a light that provides a visual flash and/or a beeper that provides an audio sound as an indication that the scanner is properly centred (Step 33).
e) The scanning direction is then rotated by 90 degrees, and the scan repeated in that direction until the display screen and/or the visual/audio indicators show that the scanner has been centred. This pinpoints the location of the transducer part 104 (Step 34)
f) The caretaker can then proceed with feeding of the patient though the NG tube 100 if the second end 102b is correctly located within the stomach of the patient (Step 35.

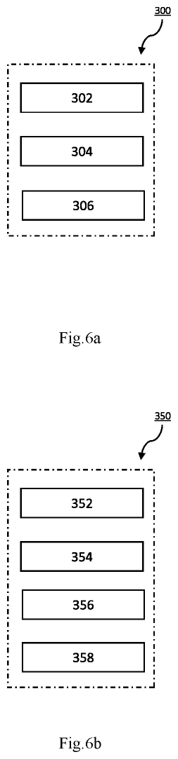

Referring to FIG. 6a, a construction method 300 in association with the NG tube 100 is shown, in accordance with an embodiment of the disclosure. Specifically, the construction method 300 can correspond to a method of construction in association with the NG tube 100, according to an embodiment of the disclosure.

The construction method 300 can, in one embodiment, include any one of a first providing step 302, a second providing step 304 and a third providing step 306, or any combination thereof.

Specifically, in one embodiment, the construction method 300 can include a first providing step 302, a second providing step 304 and/or a third providing step 306.

With regard to the first providing step 302, the tubing 102 can be provided.

With regard to the second providing step 304, the transducer part 104 can be provided.

With regard to the third providing step 306, the power supply part 106 can be provided.

The construction method 300 can further include coupling one or both of the transducer part 104 and the power supply part 106 to the tubing 102 such that the tubing 102 can carry the transducer part 104 and/or the power supply part 106.

Specifically, the tubing 102 can be shaped and dimensioned in a manner so as to be capable of carrying one or both of the transducer part 104 and the power supply part 106.

Moreover, the construction method 300 can further include coupling the transducer part 104 and the power supply part 106. Coupling can be by manner of one or both or wired coupling and wireless coupling.

The transducer part 104 may be fully sealed and embedded within a distal end of the tubing 102 by a biocompatible glue. Alternatively, the transducer part 104 may be melted with or moulded and be embedded into the material forming the tubing 102., for example the TPU material previously described. It is however also envisaged that the transducer part 104 protrude from the tubing 102, and be covered with a thin layer of sealing material, such as TPU.

Referring to FIG. 6b, a detection method 350 in association with the NG tube 100 is shown, according to an embodiment of the disclosure. Specifically, the detection method 350 can correspond to a method of detection for the purpose of verifying placement of the NG tube 100 after the NG tube 100 has been inserted/positioned in a body 100a.

The detection method 350 can include a positioning step 352, a communication step 354, a receiving step 356 and a determination step 358, or any combination thereof.

Specifically, the detection method 350 can include a positioning step 352, a communication step 354, a receiving step 356 and/or a determination step 358, according to an embodiment of the disclosure.

With regard to the positioning step 352, the detector portion(s) 100c can be positioned relative to the transducer part 104.

With regard to the communication step 354, the ultrasound-based signal(s) can be generated and communicated from the transducer part 104.

With regard to the receiving step 356, the ultrasound-based signal(s) can be received by the detector portion(s) 202,204.

With regard to the determination step 358, the received ultrasound-based signal(s) can be processed in a manner so as to generate at least one indication (audio based indication and/or visual based indication) of the location (i.e., within the body 100a) of at least one portion of the NG tube 100 (e.g., the second end 102b of the tubing 102). In one embodiment, the received ultrasound-based signal(s) can be processed by the control module 100b in an exemplary manner as discussed earlier with reference to FIG. 3a and b.

It should be further appreciated by the person skilled in the art that variations and combinations of features described above, not being alternatives or substitutes, may be combined to form yet further embodiments.

In one example, the control module 100b can be further configured to function as an external power supply source for supplying power to the transducer part 104 (i.e., so as to activate the transducer part 104). Specifically, the control module 100b can be coupled to the NG tube 100 for the purpose of supplying power to the transducer part 104. More specifically, the control module 100b can be coupled to the power supply part 106 by manner of one of both of wired coupling and wireless coupling.

In another example, the transducer part 104 can be powered by an external low voltage power pack when it is desired for the transducer part 104 to be activated.

Throughout the description, it is to be appreciated that the term ‘processor’ and its plural form include microcontrollers, microprocessors, programmable integrated circuit chips such as application specific integrated circuit chip (ASIC), computer servers, electronic devices, and/or combination thereof capable of processing one or more input electronic signals to produce one or more output electronic signals. The processor includes one or more input modules and one or more output modules for processing of electronic signals.

Unless defined otherwise, all technical and scientific terms used herein have the same meaning as is commonly understood by a skilled person to which the subject matter herein belongs.

In the foregoing manner, various embodiments of the disclosure are described for addressing at least one of the foregoing disadvantages. Such embodiments are intended to be encompassed by the following claims, and are not to be limited to specific forms or arrangements of parts so described and it will be apparent to one skilled in the art in view of this disclosure that numerous changes and/or modification can be made, which are also intended to be encompassed by the following claims.

1. A nasogastric tube positioning system comprising a nasogastric tubing having first and second ends, a power supply part located at or adjacent the first end, and a transducer part located at or adjacent the second end for transmitting or receiving an ultrasound-based signal, and a control module comprising an at least one detector portion and a processor, wherein ultrasound-based signals are transmittable between the transducer part and the at least one detector portion, the processor being adapted to record the time taken for the ultrasound-based signal to travel between the transducer part and the at least one detector portion, and to calculate a distance between the transducer part and the at least one detector portion to thereby locate the transducer part.

2. The nasogastric tube positioning system according to claim 1, wherein the transducer part is sealed within a distal tip located at the second end.

3. The nasogastric tube positioning system according to claim 2, wherein the distal tip is at least substantially dome shaped.

4. The nasogastric tube positioning system according to claim 1, wherein the power supply part is a power connector for connecting a power supply to the transducer part.

5. The nasogastric tube positioning system according to claim 1, wherein the power supply part is a power supply for supplying power to the transducer part.

6. The nasogastric tube positioning system according to claim 1, wherein the tubing comprises a first lumen extending along a length of the tubing, and one or more apertures extending through the tubing into the first lumen at or adjacent the second end thereof, the first lumen allowing fluids including food and medicine to be transferred therethrough and from the one or more apertures.

7. The nasogastric tube positioning system according to claim 6, wherein the tubing comprises a second lumen extending along the length of the tubing and separate from the first lumen, the second lumen supporting a wiring system therein electrically connecting the power supply part to the transducer part.

8. The nasogastric tube positioning system according to claim 7, wherein the wiring system is a twisted wire pair.

9. The nasogastric tube positioning system according to claim 1, wherein a radiopaque line extends along the length of the tubing.

10. The nasogastric tube positioning system according to claim 1, wherein the control module comprises two said detector portions located a fixed distance apart.

11. The nasogastric tube positioning system according to claim 1, wherein the control module is a handheld scanner having a housing for accommodating the detector portions.

12. The nasogastric tube positioning system according to claim 1, further comprising a power supply within the housing for providing electrical power to the transducer part and the detector portions.

13. The nasogastric tube positioning system according to claim 1, further comprising a display screen for showing when the detector portions are shaped equidistant from the transducer part.

14. The nasogastric tube positioning system according to claim 1, further comprising a device for displaying a light or audio signal when the detector portions are shaped equidistant from the transducer part.

15. The detection method for a nasogastric tube positioning system according to claim 1, comprising transmitting an ultrasound-based signal between the transducer part located at an end of the nasogastric tube and at least two said detector portions, recording the time taken for the ultrasound-based signal to travel between the transducer part and each of the detector portions, calculating the distance between the transducer part and each of the detector portions, and determining when the detector portions are equidistant from the transducer part to thereby locate the transducer part.

16. The detection method according to claim 15, comprising selecting the transducer part as a transmitter of the ultrasound-based signal, with the detector portions being ultrasound receivers.

17. The detection method according to claim 15, comprising selecting the detector portions as transmitters of the ultrasound-based signal, with the transducer part being a ultrasound receiver.

18. The detection method according to claim 16 or 17, comprising scanning an abdomen of a patient in a first direction initially until the detector portions are equidistant from the transducer part, and subsequently repeating the scan in a direction that is 90 degrees from the first direction.
